(19) United States
(12) Patent Application Publication
(10) Pub. No.: US 2010/0330172 A1
(43) Pub. Date: Dec. 30, 2010

(54) CONTROLLED-RELEASE PHARMACEUTICAL FORMULATION

(75) Inventors: Brett Antony Mooney, Mt. Ommaney (AU); Panagiotis Keramidas, Carindale (AU)

Correspondence Address: SUMMA, ADDITON & ASHE, P.A., 11610 NORTH COMMUNITY HOUSE ROAD, SUITE 200, CHARLOTTE, NC, 28277, US

(73) Assignee: ALPHAPHARM PTY LTD, Carole Park (AU)

(21) Appl. No.: 12/682,799

(22) PCT Filed: Oct. 13, 2008
(86) PCT No.: PCT/AU2008/001510

§ 371 Date: Aug. 3, 2010

(30) Foreign Application Priority Data
Oct. 16, 2007 (AU) 2007905661

(51) Int. Cl. A61K 31/138; A61K 9/00; A61K 9/22; A61K 9/52; A61P 25/00; A61P 25/24

(52) U.S. Cl. 424/457; 424/484; 514/653; 424/468

(57) ABSTRACT

A pharmaceutical formulation comprising desvenlafaxine having an MMD of between about 5 μm and about 100 μm, or a pharmaceutically acceptable salt thereof, and at least one matrix rate-controlling pharmaceutically acceptable polymer, solid unit dosage form containing it, methods for preparing such a formulation and for its use to treat depression and related disorders and diseases.

### FIELD OF THE INVENTION

The present invention relates to a matrix controlled-release pharmaceutical formulation comprising O-desmethylvenlafaxine, methods for preparing such a formulation and to its use to treat depression and related disorders and diseases.

### BACKGROUND

O-Desmethylvenlafaxine has a chemical name of (+/−)-4-[2-(dimethylamino)-1-(1-hydroxycyclohexyl)ethyl]phenol and is also referred to as desvenlafaxine or ODV. Desvenlafaxine has the following chemical formula:

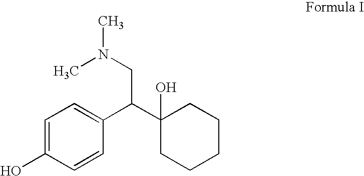

Desvenlafaxine is the major metabolite of the antidepressant venlafaxine, the latter is currently available as a hydrochloride salt as Effexor® and Effexor® SR and which is described in, for example, U.S. Pat. No. 4,761,501 and Pento, J. T., Drugs of the Future, 13(9): 839-840, 1988. Desvenlafaxine acts as a selective serotonin and norepinephrine reuptake inhibitor in the treatment of depression and other related central nervous system disorders and/or diseases. It has been suggested from in vitro studies, that desvenlafaxine is a more potent inhibitor of norepinephrine and dopamine uptake than the parent compound, racemic venlafaxine (Muth, E. A. et al, Drug Develop. Res, 23: 191-199, 1991). It has also been reported that desvenlafaxine has a half-life of about 10 hours, which is approximately 2.5 times longer that the half-life of venlafaxine (Klamerus, K. J. et al, Clin. Pharmacol., 32: 716-724, 1992).

Desvenlafaxine has been exemplified as a free base in WO 00/32555. It has also been exemplified as the fumarate salt in U.S. Pat. No. 4,535,186, as the succinate salt in U.S. Pat. Nos. 6,673,838 and 7,026,508, and in US Pat. Application 2004/0044241, and as the formate salt in U.S. Pat. No. 7,001,920 and US Pat. Application 2006/0058552.

Desvenlafaxine contains a single chiral carbon atom and thus can exist as a single enantiomer, designated as (R)-(−) or (S)-(+)-desvenlafaxine or as a racemate (i.e. 1:1 mix of R & S enantiomers). The above patents and applications exemplify the racemate. The enantiomers are exemplified in U.S. Pat. Nos. 6,342,533, 6,441,048, 6,911,479, 6,197,828 and US Pat. Applications 2004/0180952, 2002/0022662, 2002/0161055, 2003/0149112, 2004/0176468 and 2005/0256206.

Drugs in general, and antidepressants in particular can exhibit adverse events and loss of therapeutic effect after initial administration. There are many reasons for this but one cause can originate from the fluctuation in an animal or human of the plasma drug concentrations of an active substance following administration and subsequent metabolism and/or elimination from the body. These effects are sometimes referred to as peaks and troughs. Such fluctuations can be overcome by administration of the active substance in a controlled-release or sustained-release dosage form. In this manner, the active substance is more slowly administered to the body over much longer period of time. This then means that not as much active substance is available at any given time for the body to absorb. However, the overall amount of active substance administered is the substantially the same as an immediate release dosage form. In some instances, the amount of active substance in a controlled release dosage form can be less than that required in an immediate release dosage form and still achieve a comparable therapeutic effect. This can be due to the half-life of the active substance and elimination thereof from the body.

Examples of controlled-release dosage forms of antidepressants can be illustrated by marketed products such as Paroxetine CR, Venlafaxine SR, Fluoxetine Weekly, Bupropion XL, Bupropion SR, Duloxetine delayed-release and Gepirone ER. Similarly, other active substances are marketed in a controlled-release, sustained-release or extended-release dosage form. Examples include Metformin XR, Naproxen DR, Carbamazepine MR, Cefaclor CD, Diclofenac Sodium EC, Felodipine, Nifedipine CR, Omeprazole EC, Lansoprazole DR, Potassium Chloride SR, Sodium Valproate EC, Tramadol SR, Verapamil SR and the like.

These examples illustrate various methods to achieve the control over the rate of bioavailability of the active substance in the body. These control methods include rate-controlling polymer matrix systems, enteric coating systems, semipermeable water insoluble polymer coating systems, other rate-controlling polymer coating systems and mixtures thereof.

Other means to effect the rate of bioavailability of an active substance include changing its particle size or adding a surfactant. By reducing the particle size, the effective surface area of the particle is greatly increased allowing for a faster dissolution rate. The addition of a surfactant can also effect the solubility of the active substance and hence change its dissolution rate.

### SUMMARY OF THE INVENTION

In one aspect the present invention provides a matrix controlled-release pharmaceutical formulation comprising desvenlafaxine, or a pharmaceutically acceptable salt thereof, having an MMD of between about 5 μm and about 100 μm, and at least one rate-controlling pharmaceutically acceptable polymer.

In another aspect the present invention provides a solid unit dosage form comprising a matrix controlled-release formulation as described herein.

In a further aspect the present invention provides a method of preparing a matrix controlled-release formulation comprising admixing desvenlafaxine, or a pharmaceutically acceptable salt thereof, having an MMD of between about 5 μm and about 100 μm, with at least one rate-controlling pharmaceutically acceptable polymer.

In a still further aspect the present invention provides the use of a matrix controlled-release formulation as described herein, or a solid dosage form comprising the formulation, for the treatment of depression and related central nervous system disorders and diseases.

In a still further aspect the present invention provides the use of a matrix controlled-release formulation as described herein in the manufacture of a medicament for the treatment of depression and related central nervous system disorders and diseases.

In a still further aspect the present invention provides a method for the treatment of depression and related central nervous system disorders and diseases by administering a therapeutically effective amount of a matrix controlled-release formulation or a solid unit dosage form as described herein.

### BRIEF DESCRIPTION OF THE DRAWING

FIG. 1 is the in vitro dissolution profiles of embodied formulations of the present invention when measured using a USP II apparatus at 50 rpm in pH 6.8 phosphate buffer.

### DETAILED DESCRIPTION

In matrix controlled-release formulations, the rate of dissolution of the active substance is generally not expected to be the limiting factor. The swelling rate of the polymer(s) involved and the rate of water permeability through the matrix polymer are generally considered to be the controlling factors on the amount of active substance released from a matrix controlled-release formulation. Surprisingly the inventors have found that the particle size of desvenlafaxine, or a pharmaceutically acceptable salt thereof, in a matrix controlled-release formulation has an effect on the rate of release of desvenlafaxine from such a formulation and thus the bioavailability of the substance to the body.

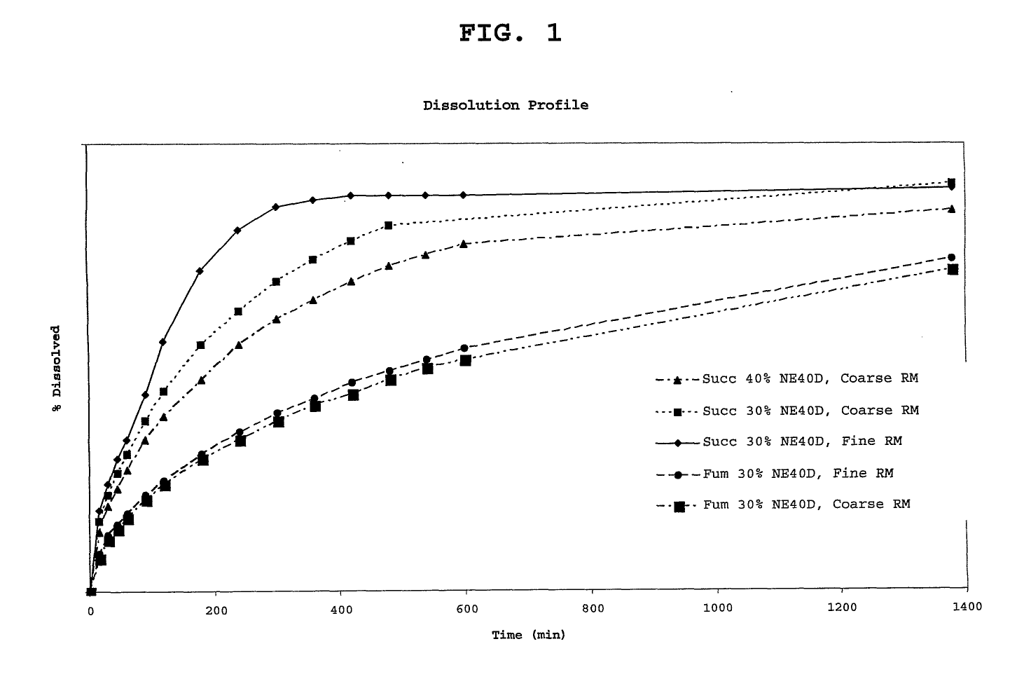

This effect has been seen as separate from a change in an amount of the rate-controlling polymer. FIG. 1 displays the result of similar formulae for batches made with 30% w/w of Eudragit NE40D or 40% w/w of Eudragit NE40D based on the total weight of the formulation and either fine or coarse desvenlafaxine succinate and fumarate. As can be seen in the FIGURE, a change from 40% to 30% of Eudragit NE40D showed an increase in the amount of desvenlafaxine dissolved at similar time points. The change from coarse to fine desvenlafaxine succinate showed at least a similar level of change in dissolution as the change from 40% to 30% of Eudragit NE40D. Thus, it can be seen that the change of coarse to fine desvenlafaxine succinate has shown a marked increase in the amount of desvenlafaxine dissolved at similar time points.

Also, a change of coarse to fine desvenlafaxine fumarate has shown an approximately similar increase in the amount of desvenlafaxine dissolved. Also shown in FIG. 1 is a difference in the solubility of the succinate salt compared to the fumarate salt.

The MMD of the particle size of desvenlafaxine, or a pharmaceutically acceptable salt thereof, in the matrix controlled-release formulation of the invention is between 5 and 100 μm, preferably between 10 and 75 μm and more preferably 20 and 50 μm.

The “mass mean diameter” or “MMD” refers to the median particle diameter based on mass (i.e. the particle diameter where one half of the mass of particles is contributed by particles with a lesser diameter and one half of the mass of particles is contributed by particles with a greater diameter) and can be measured using various commonly available methods such as measurement using light (eg. light-scattering methods or turbidimetric methods), sedimentation methods (eg. pipette analysis using an Andreassen pipette, sedimentation scales, photo-sedimentometers or sedimentation in a centrifugal force), pulse methods (eg. Coulter counter), or sorting by means of gravitational or centrifugal force.

There are various known methods for the control of the particle size of substances including reduction by comminution or de-agglomeration by milling and/or sieving, or particle size increase by agglomeration through granulation, blending or a mixture thereof. These methods use commonly available equipment and/or methods for the reduction or increase of the particle sizes of material. However, these techniques do not allow for the production of a substance with a very narrow, reproducible and consistent distribution of particle size without the need to reprocess, rework or destroy those particles outside of the required distribution. Thus, these processes can be time consuming and costly if reworking of the material under the desired size is not able to be performed. In those circumstances, it is common for the fine material to be destroyed or reprocessed.

As used herein, the term “pharmaceutically acceptable salts” refers to salts prepared from pharmaceutically acceptable non-toxic acids, including inorganic and organic acids. Suitable non-toxic acids include inorganic and organic acids such as acetic, benzenesulfonic, benzoic, camphorsulfonic, citric, ethenesulfonic, formic, fumaric, gluconic, glutamic, hydrobromic, hydrochloric, isethionic, lactic, maleic, malic, mandelic, methane-sulfonic, mucic, nitric, pamoic, pantothenic, phosphoric, succinic, sulfuric, tartaric, p-toluenesulfonic acids and the like. Particularly suitable are formic, fumaric, hydrobromic, hydrochloric, phosphoric, succinic and sulfuric acids and most particularly the fumaric and succinic acids.

Polymers that are incorporated into the matrix of a pharmaceutical dosage form and that control the rate of release of an active substance are well known in the pharmaceutical art. The matrix controlled-release polymer may suitably be selected from standard commercially available controlled-release polymers known in the art, such as the following agents: polyvinyl acetate (PVA) & polyvinylpyrollidone (PVP) copolymer such as Kollidon® SR; polyvinylpyrollidone such as Povidone K90; methacrylic/methacrylate polymers and copolymers such as Eudragit® RL, Eudragit® RS and Eudragit® NE40; celluloses such as ethylcellulose and hydroxypropyl methylcellulose such as Hypromellose K100MCR; and polyethylene oxide polymers such as POLYOX™.

A pharmaceutical formulation may include other non-active substances or excipients. These are well known in the art. Such excipients include but are not limited to those substances that acts as fillers or diluents, lubricants, flow aids or glidants, surfactants, binders and disintegrants. The lubricant can be any type typically used in art of pharmaceutical formulations. The inventors found that magnesium stearate performed particularly well.

The inventors have also found in certain embodiments of the invention, that the inclusion of a disintegrant provides formulations according to the invention having particularly advantageous properties. Whilst not wanting to be held to any particular theory, it is believed that the inclusion of a disintegrant into the matrix rate-controlled formulation of the present invention acts to establish channels through the matrix as it swells in vivo. It is believed that as the formulation contacts the gastrointestinal fluids, the uptake of this fluid acts to cause the matrix rate-controlling polymer to swell. Additionally, the disintegrant acts to attract and draw in that fluid into the matrix. The fluid then dissolves the disintegrant leaving behind channels or pores that pass through the matrix. These channels allow further fluid to pass throughout the entire formulation more rapidly. This allows for the

Examples of disintegrants include crospovidone, sodium starch glycollate and croscarmellose sodium type A. Other ingredients that can act as a disintegrant include L-HPC, HPMC and other swelling polymers, depending on the amounts used. It will be understood by the skilled artisan that any disintegrant may be used in the exploitation of the invention, however particularly preferred is the use of crospovidone, a commercially available disintegrant. It is also well within the skill-set of the skilled artisan to determine the amount of disintegrant needed. However in certain embodiments of a matrix controlled-release formulation according to the invention, approximately 1 to 15% by weight is preferred, particularly 2 to 10% most preferably 3 to 5%.

Examples of further excipients that can be utilised include lactose monohydrate as a diluent and silica colloidal anhydrous as a glidant. Other well known excipients can be used in the formulations of the invention for their common uses.

Alternatively, units in all embodiments of the invention may be coated. This coating may be functional and/or cosmetic in application. A functional coating is one that imparts some action on the dosage form, such as an enteric coating that delays release until a predetermined pH level is reached in the GI tract, a semipermeable, water insoluble coating for osmotic delivery systems, a controlled-release coating that impedes the delivery of the drug to a specific rate of release and thus deliver the active substance over a greater period of time or a taste-masking coating to protect the patient from bad tasting, bitter or otherwise unpalatable active substances. Cosmetic coatings are used to improve the appearance of the tablets and/or to aid in identification or differentiation from other products. A product can consist of more than one coating and can use intermediate or sub-coat layers to separate the core or coating layer from subsequent coating layers.

The pharmaceutical formulations of the invention can be incorporated into various pharmaceutical dosage forms for administration such as tablets, capsules, granules, pellets or beads. These formulations can be manufactured by methods well known in the art of pharmaceutical manufacture. Granules can be made by wet granulation, dry granulation or direct compression techniques. Tablets can be made by blending these granules with other pharmaceutically acceptable excipients followed by compression and optionally coated. Capsules can be made by blending granules with other pharmaceutically acceptable excipients followed by encapsulation. Pellets and beads can be made by coating nonpareils with active substance or extrusion and spheronisation.

The pharmaceutical formulations of the invention are useful in the treatment of depression and related central nervous system disorders or diseases such as anxiety, generalized anxiety disorder, social anxiety disorder, panic disorder, post traumatic stress disorder, premenstrual dysphoric disorder, vasomotor flushing, agoraphobia, borderline personality disorder, attention deficit hyperactivity disorder, autism, obsessive compulsive disorder, schizophrenia, anorexia nervosa, bulimia nervosa, Tourette's syndrome, Shy-Drager syndrome, Raynaud's syndrome, Parkinson's disease, cocaine and alcohol addiction, sexual dysfunction, obesity, chronic fatigue syndrome, urinary incontinence, fibromyalgia, pain and epilepsy.

As used throughout this specification and the appended claims, the terms “sustained or extended release”, “prolonged release”, and “controlled release”, as applied to drug formulations, have the meanings ascribed to them in “Remington's Pharmaceutical Sciences,” 18^{th} Ed., p. 1677, Mack Pub. Co., Easton, Pa. (1990). Sustained or extended release drug systems include any drug delivery system which achieves the slow release of drug over an extended period of time, and include both prolonged and controlled release systems. If such a sustained release system is effective in maintaining substantially constant drug levels in the blood or target tissue, it is considered a controlled release drug delivery system. If, however, a drug delivery system is unsuccessful at achieving substantially constant blood or tissue drug levels, but nevertheless extends the duration of action of a drug over that achieved by conventional delivery, it is considered a prolonged release system.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It will be clearly understood that, although a number of prior art publications are referred to herein, this reference does not constitute an admission that any of these documents form part of the common general knowledge in the art, in Australia or in any other country.

### Example

The particle size of desvenlafaxine utilised in the following formulations are set out in Table 1.

TABLE 1
Desvenlafaxine particle size

| Grade | MMD |
|---|---|
| Coarse succinate | 186 μm |
| Fine succinate | 37 μm |
| Coarse fumarate | 184 μm |
| Fine fumarate | 21 μm |

The formulation used in this experiment for the coarse and fine batches is as set out in Table 2.

TABLE 2
Formulation

| Ingredients | 40% NE40D, | 30% NE40D, | 30% NE40D, | 30% NE40D, | 30% NE40D, |
|---|---|---|---|---|---|
| (mg/tablet) | Coarse RM | Coarse RM | Fine RM | Coarse RM | Fine RM |
| ODV-Succinate | 113.81 | 113.81 | 113.81 | — | — |
| ODV-Fumarate | — | — | — | 113.18 | 113.18 |
| Calcium Hydrogen | 137.19 | 147.19 | 147.19 | 147.82 | 147.82 |
| Phosphate | | | | | |
| Eudragit NE40D | 40 | 30 | 30 | 30 | 30 |
| Water | qs | qs | qs | qs | qs |
| Talc | 6 | 6 | 6 | 6 | 6 |
| Magnesium Stearate | 3 | 3 | 3 | 3 | 3 |
| TOTAL | 300 | 300 | 300 | 300 | 300 |

The resulting in vitro dissolution profiles of these batches as tested using a USP II apparatus (paddles) at 50 rpm in pH 6.8 phosphate buffer are set out in Table 3.

TABLE 3
Dissolution Results

| | Succinate Salt | | | Fumarate Salt | |
|---|---|---|---|---|---|
| | 40% NE40D, | 30% NE40D, | 30% NE40D, | 30% NE40D, | 30% NE40D, |
| TIME | Coarse RM | Coarse RM | Fine RM | Coarse RM | Fine RM |
| 0 | 0 | 0 | 0 | 0 | 0 |
| 15 | 16 | 19 | 22 | 9 | 10 |
| 30 | 23 | 26 | 29 | 14 | 15 |
| 45 | 28 | 32 | 36 | 17 | 18 |
| 60 | 33 | 37 | 41 | 20 | 21 |
| 90 | 41 | 46 | 53 | 25 | 26 |
| 120 | 47 | 54 | 67 | 29 | 30 |
| 180 | 57 | 66 | 86 | 36 | 37 |
| 240 | 66 | 75 | 97 | 41 | 43 |
| 300 | 73 | 83 | 103 | 46 | 48 |
| 360 | 78 | 89 | 105 | 50 | 52 |
| 420 | 83 | 94 | 106 | 53 | 56 |
| 480 | 87 | 98 | 106 | 57 | 59 |
| 540 | 90 | N/A | 106 | 60 | 62 |
| 600 | 93 | N/A | 106 | 62 | 65 |
| 1380 | 102 | 109 | 108 | 86 | 89 |

1. A matrix controlled-release pharmaceutical formulation comprising desvenlafaxine, or a pharmaceutically acceptable salt thereof, having an MMD of between about 5 μm and about 100 μm and at least one matrix rate-controlling pharmaceutically acceptable polymer.

2. The formulation of claim 1 wherein the pharmaceutically acceptable salt of desvenlafaxine is a non-toxic acid-addition salt.

3. The formulation of claim 1 wherein the pharmaceutically acceptable salt of desvenlafaxine is selected from one of the formate, fumarate or succinate salt.

4. The formulation of claim 1 wherein the pharmaceutically acceptable salt of desvenlafaxine is the fumarate salt.

5. The formulation of claim 1 wherein the pharmaceutically acceptable salt of desvenlafaxine is the succinate salt.

6. The formulation of claim 1 wherein the MMD of desvenlafaxine, or a pharmaceutically acceptable salt thereof, is between about 10 μm and about 75 μm.

7. The formulation of claim 6 wherein the MMD of desvenlafaxine, or a pharmaceutically acceptable salt thereof, is between about 20 μm and about 50 μm.

8. A solid unit dosage form comprising the matrix controlled-release formulation of claim 1.

9. The solid unit dosage form of claim 8 which is selected from tablets, capsules, granules, pellets or beads.

10. The solid unit dosage form of claim 8 which is selected from tablets or capsules.

11. Method of preparing a matrix controlled-release formulation comprising admixing desvenlafaxine, or a pharmaceutically acceptable salt thereof, having an MMD of between about 5 μm and about 100 μm with at least one matrix rate-controlling pharmaceutically acceptable polymer.

12. (canceled)

13. (canceled)

14. A method for the treatment of depression and related central nervous system disorders and diseases by administering a therapeutically effective amount of the matrix controlled-release formulation of claim 1.

15. A method for the treatment of depression and related central nervous system disorders and diseases by administering a therapeutically effective amount of the solid unit dosage form of claim 8.
